(19) United States
(12) Patent Application Publication
(10) Pub. No.: US 2024/0382255 A1
(43) Pub. Date: Nov. 21, 2024

(54) Techniques For Delivering Laser Energy To A Target Site Of An Organism

(71) Applicant: Industrial Laser Machines, LLC, Winter Park, FL (US)

(72) Inventor: Dwight Evans Kimberlin, Winter Park, FL (US)

(21) Appl. No.: 18/198,925

(22) Filed: May 18, 2023

(51) Int. Cl. A61B 18/22

(52) U.S. Cl. CPC A61B 18/22; A61B 2018/2005; A61B 2018/20553; A61B 2018/2266

(57) ABSTRACT

Techniques are disclosed for delivering laser radiation to a target site in an organism. Preferably, pulsed laser radiation is delivered to the target site using a hypodermic needle. Depending on the embodiment, a focusing lens is used to focus the laser radiation into the needle shaft through which the laser light is propagated and delivered/transferred to the target site. The hypodermic needle may be a standard hypodermic needle or a modified hypodermic needle containing a lens. Various types of adapters are disclosed that attach to standard optical connectors at one end and to the standard connections of a hypodermic needle at the other end. In variations, the laser-delivery hypodermic needles of the instant design are self-contained and wirelessly controlled. The embodiments can be practiced to benefit a variety of use-cases in the medical, healthcare, life sciences and other fields.

### FIELD OF THE INVENTION

This disclosure relates generally to medical devices, and more particularly to devices capable of delivering laser/electromagnetic radiation to target sites within an organism.

### BACKGROUND

The evolution of laser therapy has reduced the need for open surgical and invasive procedures to treat various medical conditions and ailments. Furthermore, traditional surgical procedures, both therapeutic and diagnostic, for pathologies located within the body can cause significant trauma to the intervening tissues. These procedures often require a long incision, extensive muscle stripping, prolonged retraction of tissues, denervation and devascularization of tissue.

The above traditional procedures can require long operating times followed by extensive post-operative recovery time due to the destruction of tissue during the surgical procedure. In some cases, these invasive procedures lead to permanent scarring and pain that can be more severe than the pain leading to the surgical intervention. The availability of laser technology has simplified the treatment of various ailments as well as simplified post-operative protocols.

There is plenty of prior art that teaches such laser technology-based treatments and therapies. U.S. Pat. No. 11,491,345 B2 to Rogers et al. describes devices, assemblies, and their systems and methods for targeting one or more sites with electromagnetic radiation. The devices, assemblies and systems are operationally configured to transform and convey electromagnetic radiation to one or more targeted sites. The devices, assemblies and systems may also convey one or more fluids or fluid solutions to the one or more targeted sites.

U.S. Pat. No. 11,547,446 B2 to Kienzle et al. teaches a fully integrated sterilizable one-time-use disposable tissue visualization device and methods for using such devices. Preferred embodiments of their disclosure facilitate the visualization of an internal tissue site while causing minimum of damage to the surrounding tissue. Further preferred embodiments may allow for the delivery of fluids and other treatment to an internal tissue site.

U.S. Patent Publication No. 2017/0189711 A1 to Shur et al. teaches medical devices capable of delivering and/or sensing electromagnetic radiation within an organism body. Placement of a location for delivering and/or sending the electromagnetic radiation is done through the skin of the organism body. Devices described include a needle with a wave-guiding structure configured to direct the electromagnetic radiation to/from a location within the organism body.

U.S. Patent Publication No. 2022/0401027 A1 to Mowery et al. describes a precision, forward-looking, image-guided, diagnostic and therapeutic surgical probe and needle insert for microsurgery. In support of imagery, neurology, neurosurgical procedures, and ophthalmic surgical applications, there is an introducer needle (stylet), a fiber carrier, a therapeutic conduit, and a spirographic method for scanning a target and associated algorithms to create and render a reconstructed image for display to a physician in real-time or near real-time.

The probe implements Optical Coherence Tomography (OCT) to provide high-resolution extended imagery of an intended therapeutic or target tissue. A separate therapeutic conduit provides surgical access for therapeutic devices such as a cutting or ablation laser, an RF electrode for locally heating tissue, a lumen for local injection of neurolytics/paralytics, placement of electrodes for neuromodulation, and deployment of a micro-endoscopic imaging tool. A third working channel supports the delivery of neurolytic and other fluids.

U.S. Patent Publication No. 2014/0121637 A1 to Boyden et al. teaches a system and methods for generating an injection guide, which includes receiving one or more digital images of a body region of an individual. The images include one or more physical registration landmarks, for generating at least one digital representation of the body region using the one or more digital images. The at least one digital representation includes one or more digital registration landmarks corresponding to the one or more physical registration landmarks on the body region.

Further added are one or more digitally registered injection sites to the at least one digital representation of the body region in an injection treatment pattern. The one or more digitally registered injection sites are registered relative to the one or more digital registration landmarks, and generate one or more output signals. These output signals have information for controlling one or more controllable light-emitting elements to illuminate a location on a surface of the body region of the individual in a location corresponding to at least one of the one or more digitally registered injection sites.

U.S. Pat. No. 11,311,336 B1 to Djeu et al. describes a hand piece for delivering energy from a laser to a target through a fiber tip including an elongated handle having a proximal end and a distal end. An axial bore traverses the handle along a longitudinal axis thereof. A fiber tip holding mechanism is fixed within the axial bore of the handle and includes a forward section fixed with the handle, a spring, and a rear section that includes a hollow collet projecting forward therefrom that traverses the spring and the forward section. The forward section is slidable rearward towards the rear section to compress the spring in a rear position. The spring urges the forward section forward into a forward position. A chuck is fixed with the forward end of the fiber tip holding mechanism and includes a tapered sleeve and two or more oppositely tapered jaws.

U.S. Patent Publication No. 2014/0236023 A1 to Day et al. teaches a probe, such as a spectroscopic probe, for enabling a fluid or tissue sample to be tested in situ. The probe includes a conduit, such as a hypodermic needle, that can be inserted into a test subject and a wave coupling arranged to direct electromagnetic radiation, such as light, from an energy source to the sample and/or from the sample to a receiver for analysis. The receiver may comprise a Raman spectroscope. The probe may include a carriage that can be used to move at least some of the optical coupling towards and away from the insertion tip of the conduit. The probe may include a pressure modifier that can be used to draw fluid into or expel fluid from the conduit.

Furthermore, traditional state of the art to deliver therapeutic laser energy internally to the body is by making a surgical incision to expose the area to be treated, or using an endoscope, or using external radiation from outside the body. These approaches are limited by the depth of penetration and precision of the applied radiation. Prior art for alleviating ruptured disks in the vertebrae is to fuse the vertebrae together or to insert a metal rod into the spine.

Similarly, prior art laser techniques for treating ruptured or damaged disks require incisions to be made to get the laser energy inside the disk. A less effective present method uses indirect stimulation from outside the body. While tumors and cysts can be destroyed by laser energy, an incision still has to be made to expose them to the laser energy. Inoperable tumors currently cannot be treated this way.

Prior art laser therapy being used for the skin is another example of the ability of laser light to rejuvenate cells, however traditional techniques require invasive methods. Optogenetics is a rapidly growing field currently limited by not having an easy way to deliver the laser energy where needed without surgery and/or optical implants. Also, current acupuncture treatments insert needles at various specific points on the body. Sometimes electricity is applied to the needles. Laser Angioplasty uses invasive catheters to remove plaque from arterial walls. These treatments can also benefit from minimally invasive laser techniques that are currently absent in the art.

What is needed and absent from the prior art are techniques that will enable effective delivery of laser radiation to any part of an organism's body without requiring incisions or other substantially invasive methods.

### OBJECTS OF THE INVENTION

In view of the shortcomings of the prior art, it is an object of the invention to provide techniques for delivering laser energy to any target site of an organism without requiring incisions.

It is also an object of the invention to deliver laser energy by utilizing hypodermic needles that are minimally invasive and can reach any target site of the organism's body without collateral damage to the surrounding tissues.

It is also an object of the invention to utilize pulsed laser radiation for delivering laser energy by an instant laser-delivery hypodermic needle to the target site.

It is also an object of the invention to provide various adapters and mating interfaces that will enable off-the-shelf medical and optical components to be used for laser-delivery hypodermic needles with no or minimal modifications.

It is also an object of the invention to enable laser-delivery hypodermic needs to be wearable or indwelling for extensive periods of time.

It is also an object of the invention to provide for the laser-delivery hypodermic needles to be self-contained and wirelessly controlled.

Still other objects and advantages of the invention will become apparent upon reading the summary and the detailed description in conjunction with the drawing figures.

### SUMMARY OF THE INVENTION

A number of objects and advantages of the invention are achieved by apparatus and methods for a laser-delivery hypodermic needle that delivers laser energy to a target site of, in or within an organism. The organism may be a member of the Animal Kingdom (also known as Kingdom Animalia) or Plant Kingdom (also known as Kingdom Plantae). Preferably, the laser energy is in the form of pulsed laser radiation that is generated by a pulsed laser source. The pulsed laser radiation is then carried by an optical fiber to a hypodermic needle. The optical fiber is preferably connected to said hypodermic needle via an adapter.

The hypodermic needle is inserted into the organism towards a target site of interest. The insertion is preferably performed with the aid of an imaging or visual guidance system that allows a human operator, such as a doctor, to visually observe the needle as it is inserted into the organism. Rather than a human operator, the insertion may also be performed by a robot. In this manner, the doctor can avoid damaging any other tissues or organs within the organism while the needle traverses through the organism to its target site.

Pulsed laser energy carried to the hypodermic needle light-pipes through the needle shaft due to total internal reflection and is delivered to the target site in the organism. It is thus desirable to have the shaft internally polished to facilitate the above light-piping and total internal reflection of the laser radiation.

In a preferred embodiment, there are one or more lenses embedded in the hypodermic needle itself, or the adapter mentioned above. The lens or lenses focus the laser beam or radiation inside the shaft. The focusing in the needle shaft enables the radiation to travel through more efficiently and to be able to carry laser energy to the target site more efficiently.

Depending on the variation, the lens(es) are also useful for preventing contamination of the target site and consequently the organism by blocking any bodily fluid of the organism from coming in contact with the optical fiber or the laser source. Alternatively or in addition, the needle shaft has a filling that prevents such contamination. The filling may be entirely or partially made of glass or a polymer or any other suitable material that allows the laser radiation to pass through it.

The optical fiber preferably connects to the adaptor by utilizing one of a variety of optical fiber connectors or connects available in the industry. These include but are not limited to a bayonet connector, a friction lock, a magnetic connector, a SubMiniature version A (SMA) Connector, a Straight Tip (ST) connector, a Subscriber Connector (SC), a Miniature Unit (MU) connector, a Fiber Channel (FC) connector, a Mechanical Transfer Registered Jack (MTRJ) connector, a Lucent Connector (LC) and an E2000 connector. Similarly, the needle-hub is connected to the adapter via one of a luer lock, a slip-tip connector, a catheter tip connector, a threaded connector and an eccentric tip connector, among others.

Depending on the embodiment, the needle shaft may be bent to accommodate the varying needs of an application. More specifically, the needle shaft may be bent at any angle in the range of 0 to 179 degrees to reach a variety of different target sites in the organism in a manner least destructive to the surrounding tissues and organs.

Also depending on the embodiment, the bevel of the hypodermic needle may have one of a variety of different forms to fit the needs of a given application. For example, the bevel end may be axisymmetric, in which case the laser beam emanating from the bevel will have an omnidirectional pattern. Alternatively, the bevel may be asymmetric, in which the case the laser beam emanating from the bevel will have a unidirectional pattern.

The present technology is suitable for a variety of different medical and healthcare applications. In one preferred embodiment, the present design is used for acupuncture treatment. For acupuncture, the shaft preferably has a micro-gauge, although that is not a requirement. Typically, more than one instant hypodermic needles will be used for acupuncture.

The needs for an acupuncture treatment setup can be met by a variety of different variations based on the instant principles. In a preferred acupuncture variation, the pulsed laser radiation generated by the pulsed laser source is first transmitted through a beam splitter. A beam splitter splits the incident laser beam into multiple laser beams. Each such resultant laser beam has less power than the incident beam, making the low-power laser beams especially attractive for acupuncture.

Each laser beam outputted by the beam splitter is then carried by an individual fiber optic cable to a corresponding hypodermic needle of the present design. Each such instant acupuncture hypodermic needle or simply instant acupuncture needle is then inserted at a desirable target site in the organism.

Having a beam splitter is not a requirement for acupuncture, however. Therefore, in alternative variations, each instant acupuncture needle may be powered by its own appropriately-powered laser source that is operably connected to the needle. In one interesting variation of the present application, the laser source is integrated with the instant laser-delivery device. Preferably, this leads to a low-cost device that is disposable after one or more uses. Preferably, such a device is manufactured using single-mold manufacturing techniques.

There are many other interesting and useful applications of the present technology for healthcare, medicine, life-sciences and other disciplines. Thus, depending on the variation, the target site in the organism may be delivered laser radiation using the present techniques for a variety of reasons. These include but are not limited to treatment, growth, repair, rejuvenation, etc. Therefore, the target site of interest in the organism of interest may include but is not limited to the following: a tumor, a cyst, a polyps, a joint, a cartilage, a spinal cord disc, one or more stem cells, an acupuncture site, an optogenetic treatment site, etc.

In yet another highly preferred embodiment, the pulsed laser-delivery hypodermic needle of the instant design is used as a wearable or as an indwelling device. Exemplary indwelling devices include a catheter, a cannula, etc. The indwelling device may stay connected to the instant organism for an extended period of time. When used as an indwelling/wearable device, it is preferable for the instant laser source and needle to be integrated with each other. Such an objective is advantageously achieved with a single-mold design and the instant indwelling device is thus preferably disposable after one or more uses.

In a highly preferred embodiment, the pulsed laser radiation delivered to the target site is controlled wirelessly. What this means is that the duration and/or the number and/or the power of the laser pulses delivered to the target site may be controlled from a control system that is not physically connected to the instant hypodermic needle. Using such an apparatus and its methods, a human such as a doctor/physician can precisely control the delivered laser radiation remotely and for an extended period of time. For this purpose, a battery, a wireless adapter/controller e.g. a Wifi or Bluetooth adapter as well as the laser source are integrated with the instant laser-delivery device.

The present embodiment can thus enable applications that allow for an instant wearable device to remain attached to the instant organism for an extended period of time. Exemplary uses of such a device may be for chronic or acute pain treatment, rejuvenation, repair of target site or tissues, among others. The wireless technology used in the present embodiments includes Wifi, Bluetooth, Near Field Communication (NFC), ZigBee or any other remote wireless technology. Such a technology may utilize electromagnetic or other waves, such as acoustic waves, etc.

Depending on the variation, the laser source used in the present design may be a solid-state laser or a gas laser. Exemplary laser source technologies available in the industry that may be used within the scope of the present principles include a laser diode and fiber lasers.

Furthermore, a variety of imaging techniques may be harnessed for properly and accurately directing the instant laser hypodermic needle to the target site without damaging surrounding parts of the organism. Such imaging techniques may be online i.e. used in real-time as visual-aid for an operator or a robot that inserts the needle or the organism. The imaging techniques may also be offline such as X-rays or Computerized Tomography (CT) or Magnetic Resonance Imaging (MRI) scans that the operator/robot uses/ingests beforehand prior to inserting the needle. The imaging techniques may be screen based, hologram based or even paper based depending on the type of application of the present technology.

Clearly, the system and methods of the invention find many advantageous embodiments. The details of the invention, including its preferred embodiments, are presented in the below detailed description with reference to the appended drawing figures.

### BRIEF DESCRIPTION OF THE DRAWING FIGURES

FIG. 1 shows a laser-delivery hypodermic needle based on the instant principles that uses an SMA optical connector, has a straight shaft and does not require an adapter.

FIG. 2 is a variation of the embodiment of FIG. 1 with a bent or angled shaft.

FIG. 3 shows a laser-delivery hypodermic needle based on the instant principles that uses an ST optical connector, has a straight shaft and does not require an adapter.

FIG. 4 is a variation of the embodiment of FIG. 3 with a bent or angled shaft.

FIG. 5A-B show an embodiment that uses an adapter that embeds a lens and attaches to a standard hypodermic needle at one end and to an SMA fiber optic connector at the other end.

FIG. 6A-B show an embodiment that uses an adapter that embeds a lens and attaches to a standard hypodermic needle at one end and to an ST fiber optic connector at the other end.

FIG. 7 show embodiments that use a standard hypodermic needle that is modified to contain a focusing lens. Two variations, one with a straight and one with an angled shaft are shown.

FIG. 8 shows a self-contained and wireless design of the instant laser-delivery hypodermic needle that is battery powered and is wirelessly controlled.

FIG. 9 shows an application utilizing the embodiment of FIG. 8 for delivering laser radiation to a target site in a human arm.

FIG. 10A shows an acupuncture application utilizing the self-contained and wireless embodiment of FIG. 8.

FIG. 10B shows an acupuncture application utilizing instant laser-delivery hypodermic needles that are cabled/wired.

FIG. 11A-B show various bevel ends that may be utilized by instant laser-delivery hypodermic needles according to the needs of an application.

FIG. 12A-C show various fillings in the shafts of instant laser-delivery hypodermic needles.

FIG. 13 shows yet another embodiment utilizing a standard hypodermic needle with a specialized optical connector attached to it via a luer lock.

### DETAILED DESCRIPTION

The figures and the following description relate to preferred embodiments of the present invention by way of illustration only. It should be noted that from the following discussion, alternative embodiments of the structures and methods disclosed herein will be readily recognized as viable alternatives that may be employed without departing from the principles of the claimed invention.

Reference will now be made in detail to several embodiments of the present invention(s), examples of which are illustrated in the accompanying figures. It is noted that wherever practicable, similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

Let us now review the techniques for the delivery of laser radiation to an organism based on the instant principles. The various embodiments taught in this disclosure can benefit from any form of electromagnetic radiation including non-laser or non-coherent electromagnetic radiation of any wavelength, including infrared light, ultraviolet light, and even X-rays, depending on the specific application requirements. However, special emphasis is paid to laser radiation or laser light as the form of electromagnetic radiation delivered to the target site.

This is because laser radiation is highly coherent. The higher the coherence, the easier it is for biological cells to use the incident radiation. Various types of living cells have shown to be able to be rejuvenated by exposure to the proper amount of energy and wavelength of laser radiation. There are many different types of laser sources that can be utilized to accrue the benefits of the present design. These include but are not limited to gas lasers, solid-state lasers, semiconductor lasers, dye lasers, fiber lasers, excimer lasers, etc.

The present techniques are agnostic to the wavelength of the laser radiation, which may be chosen based on the application. Furthermore, the various embodiments taught in this disclosure can benefit from any form of laser light, whether continuous or pulsed. However, special emphasis is paid to pulsed laser radiation to more comprehensively accrue the benefits of the present technology.

Those skilled in the art will understand that unlike a continuous or continuous-wave or steady state laser, pulsed lasers emit light in a series of pulses. The pulses have a duration and a repetition rate or frequency, whereas continuous-wave lasers emit a steady beam of light with a constant power. Pulsed lasers offer a range of advantages over continuous lasers, making them well-suited to a wide range of applications. These advantages include high peak power as well as greater precision since pulsed lasers can be finely tuned to deliver extremely short, precise bursts of energy, which can be used for delicate and precise applications as in the present embodiments. The advantages further include lower power input, better efficiency, better safety and generally lower power consumption and operating costs.

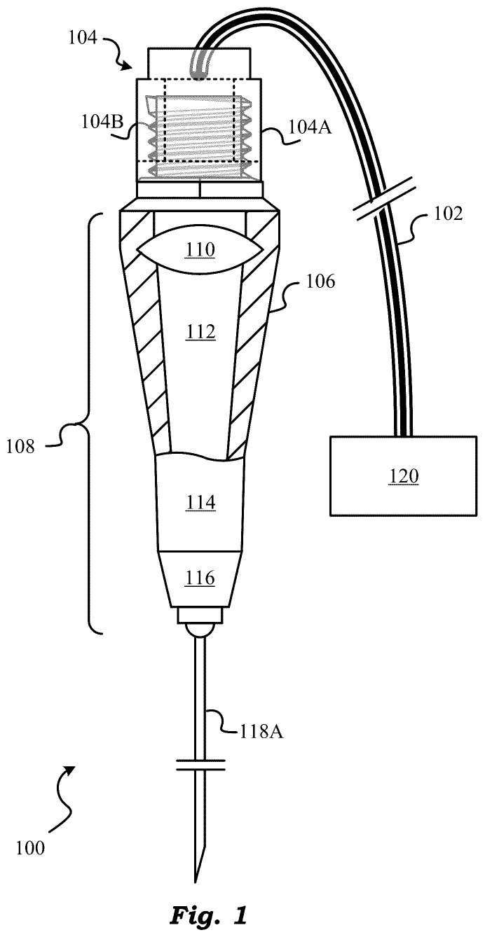

Armed with the above knowledge, let us now first review a preferred embodiment 100 shown in FIG. 1. FIG. 1 shows a laser radiation delivery or simply a laser-delivery system 100 that contains a pulsed laser source 120. Exemplarily, the pulses of laser light generated by source 120 have a duration in the range of 5 picoseconds to 500 nanoseconds. However, they may have a duration of any desired length of time and may be generated with any desired frequency by pulsed laser source 120.

As shown in FIG. 1, laser source transmits pulsed laser radiation through an optical fiber cable or optical fiber or fiber optic 102 of a desirable length. Optical fiber 102 in turn feeds into a fiber optic connector 104. The exemplary fiber optic connector 104 shown in FIG. 1 is a popular SubMiniature version A or SMA connector whose male member 104A threads over its female counterpart member 104B. In an alternative variation, the roles of the male and female members of optical connector 104 are reversed. In other words, member 104B threads around member 104A in such a variation.

Now, connector 104, specifically its female component or member 104B is attached to a body 108 of an instant hypodermic needle. Needle body 108 in turn contains a lens housing 106 in which a lens 110 is embedded or situated. Within lens housing 106 is a cavity 112 that has a vertical length that is adjustable based on where exactly lens 110 is placed according to the requirements of an application. There may be other members or portions of body 108 according to the specific manufacturing design used for the instant hypodermic needle but their shapes, dimensions and existence is not a requirement to accrue the benefits of the present principles.

In the example shown in FIG. 1, there is a member 114 and a member 116 and below which there are rectangular and semi-circular members as shown but are not explicitly marked to avoid clutter. Any number and shapes of such optional members below lens housing 106 and within needle body 108 may exist and may also be integrated together. In industry literature, these members comprise what is sometimes referred to as a needle-hub. A needle-hub in a conventional syringe is referred to as the dead space between the needle and the barrel of the syringe. The traditional needle-hub collects liquid that once drawn up cannot be plunged back out.

Based on the instant principles, a needle-hub or simply a hub refers to any member(s) of the needle between needle shaft 118A and fiber optic connector 104. Exactly which such members comprise the hub will depend on the embodiments as explained herein. Thus, in the variation shown in FIG. 1, the needle-hub consists of members 114, 116 and any other members before shaft 118A. Needle body 108 of FIG. 1 comprises the hub and lens housing 106. The instant needle-hub is preferably integrated with lens housing 106 in needle body 108 for a compact design.

Depending on the embodiment, needle shaft 118A may or may not have a filling of a suitable material. The filling, not explicitly shown in FIG. 1, may be made of glass or a polymer or any other material transmissive enough for the laser wavelengths being used. The filling may even just comprise of the bodily fluid of the organism that travels up or backfills needle shaft 118A but is transmissive enough for the wavelength of the laser to propagate through it. In any case, the laser radiation travels through the filling for delivery to the target site. Furthermore, shaft 118A may be of any length and diameter to suit the needs of a specific application.

According to the chief aspects, pulse laser radiation generated by pulsed laser source 120 travels or transmits through fiber optic cable 102 to fiber optic connector 104. It is then incident on the upper surface of lens 110 which collects the incident radiation. Based on its location in lens housing 106 and consequently on the size of cavity 112, lens 110 focuses the laser light/radiation into needle shaft 118A. The purpose of cavity 112 is to allow laser radiation emanating from the bottom surface of lens 118 to focus into the shaft 118A for the delivery of laser energy to the target site of the organism. Length of cavity 112 and placement of lens 110 can be adjusted based on the focal length of the lens and shaft 118A.

In variations, lens 110 may comprise of more than one lenses and/or mirrors and may thus be a compound lens consisting of more than one optical component. The target site in an organism of interest as taught in this disclosure may be a site within or in any part of the organism, either on or close to the surface or the skin of the organism, or deep in the interior of the organism or anywhere in between. By interior of an organism, we mean a site that is deeper than the surface or the outer skin of the organism. The organism may be a member of the Animal Kingdom (a.k.a. Kingdom Animalia) or Plant Kingdom (a.k.a. Kingdom Plantae).

Furthermore, instant laser-delivery hypodermic needles of any embodiments taught in this disclosure, including embodiment 100 of FIG. 1, are inserted into the organism and preferably guided to the target site(s) with the aid of an imaging or guidance system. A variety of imaging technologies may be harnessed for properly and accurately directing an instant laser hypodermic needle to the target site without damaging surrounding parts of the organism. Such imaging techniques may be online i.e. used in real-time as a visual-aid for an operator, such as a doctor/surgeon, or a robot that inserts the needle into the organism.

The imaging systems or visual-aid may also be offline such as an X-ray radiograph or a computerized tomography (CT) scan or a magnetic resonance imaging (MRI) scan that the operator/robot uses/ingests beforehand prior to inserting the needle. The offline imaging system may have the above-mentioned pictures/scans in physical forms or in electronic forms or both. Furthermore, the imaging systems used by the present technology may also benefit from Augmented Reality (AR) and Virtual Reality (VR) techniques.

Such AR/VR techniques allow for a real-world image or scene of the organism/patient to be superimposed with an AR image/scene in a viewing/display system, such as a headset. The superimposed, merged or combined image/scene thus obtained guides an operator, such as a doctor, to insert and advance an instant needle to an intended target site in the organism. In summary, the imaging systems/technologies/techniques used by the present embodiments may be real-time as the treatment is being performed, or offline i.e. consist of a prior snapshot of the organism. They may be viewed on a screen, as a hologram or even on paper depending on the type of application of the present technology.

This capability to minimally invasively deliver laser radiation that is preferably pulsed and controlled per the requirements of the application, to any target site within the organism body is a key contribution of the present technology. Any loss of power through the needle can be calibrated and compensated for to deliver the proper dosage at the output. Depending on the variation, shaft 118A of instant laser-delivery hypodermic needle 100 of FIG. 1, may be as long or narrow/wide as needed to safely reach the target site of the organism.

Shaft 118A is typically made out of metal, although that is not a requirement of the present design. The advantage of a metal shaft is its robustness and resistance to internal breakage. Internal breakage refers to the breaking of the shaft while it is inserted or is inside the organism, and can evidently lead to adverse consequences for the treatment. Shaft 118A may also be internally polished, i.e. its internal bore polished, to facilitate light-piping and total internal reflection of the transmitted laser radiation. In alternative variations, shaft 118A may be entirely or partially made out of glass or a polymer or any other suitably transmissive material.

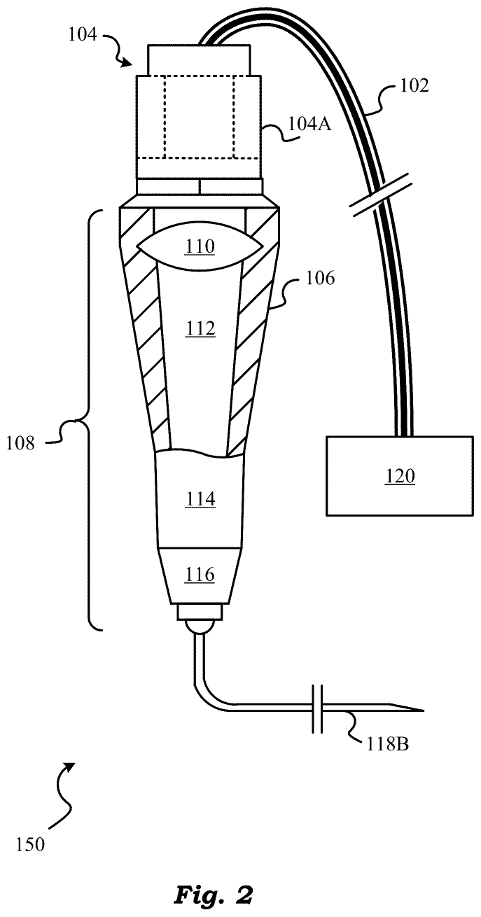

The shaft may also be bent at an angle in one or more places to effectively reach the target site without disturbing or damaging any surrounding tissue of the organism. For example, FIG. 2 shows a variation 150 of the embodiment 100 of FIG. 1 with a needle shaft 118B that is bent in one place at an angle of 90 degrees. Note, FIG. 2 shows optical connector 104 as an opaque object without showing the threads of its male member 104A and female member 104B of the above discussion.

Per above, the organism of the present teachings to who the laser energy is being delivered, and more specifically to whose target site or sites the laser energy is being delivered, may be any organism of the Animal or Plant Kingdoms. In the preferred embodiments, the organism is a human being e.g. a patient, but it may also be an animal or an animal patient, including a pet or a wild or an agricultural animal. It may also be a plant or a brush or a tree or any part thereof, such as a root or a trunk, etc.

Depending on the application, the target site may be an internal organ of the organism, a tumor, a cyst, a polyps, a joint, a cartilage, a spinal cord disc, one or more stem cells, an acupuncture site or an optogenetic treatment site in/within the organism. It may be any part of the body of the organism to which laser energy, preferably in its pulsed form, is delivered via a hypodermic needle to accrue the benefits of the present principles.

The embodiments of FIG. 1-2 show optical connectors that are exemplarily SMA connectors. However, the instant design is agnostic of the type of optical connector, presently available or to be designed in the future, that is used to connect optical fiber 102 to hypodermic needle body 108. Some of the optical connectors available in the industry within the scope of the present principles include but are not limited to a bayonet connector, a friction lock, a magnetic connector, a SubMiniature version A (SMA) Connector, a Straight Tip (ST) connector, a Subscriber Connector (SC), a Miniature Unit (MU) connector, a Fiber Channel (FC) connector, a Mechanical Transfer Registered Jack (MTRJ) connector, a Lucent Connector (LC) and an E2000 connector.

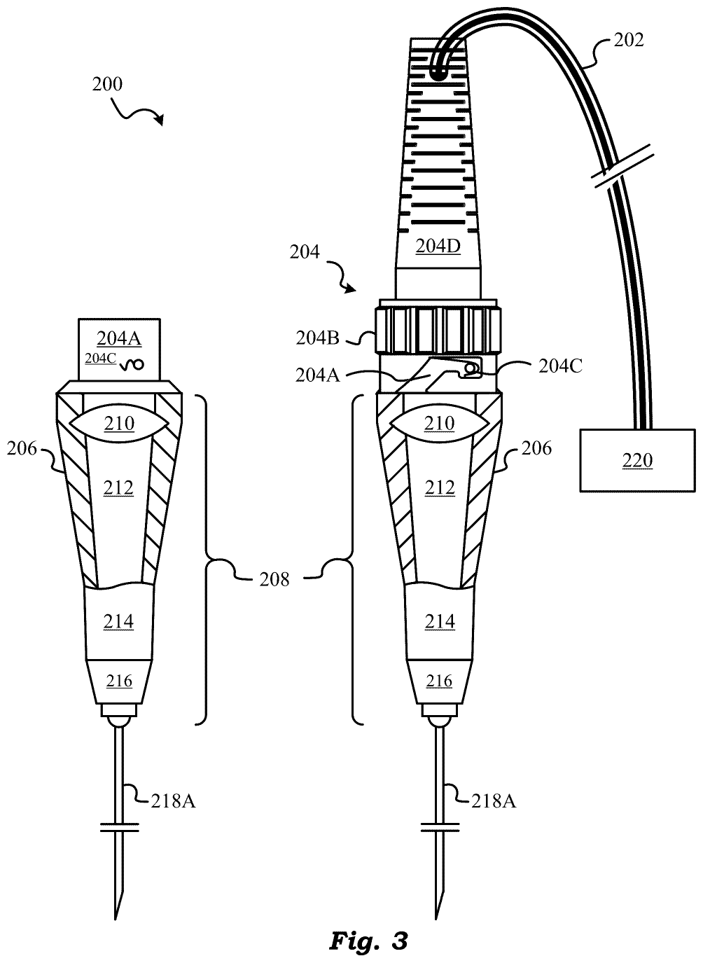

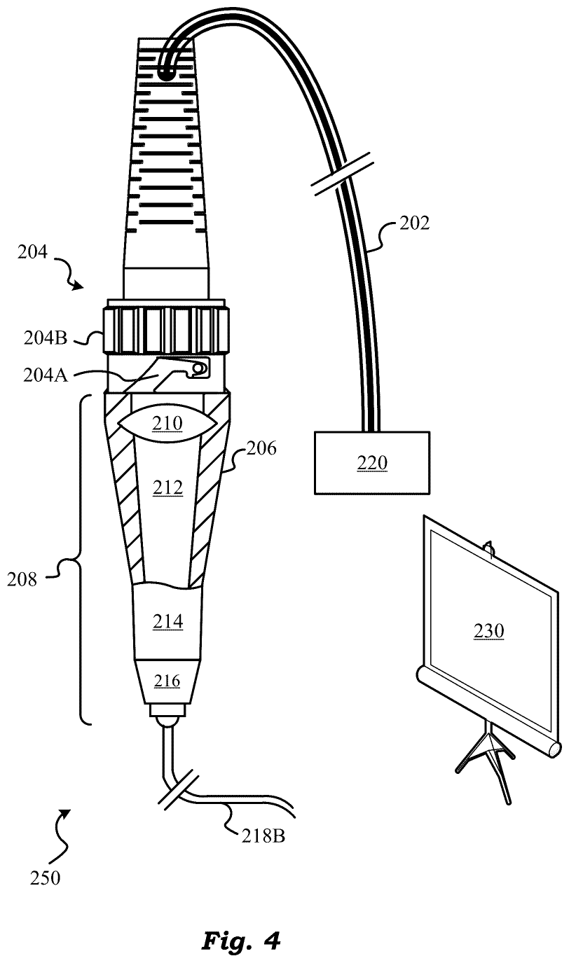

FIG. 3 and FIG. 4 explicitly show embodiments 200 and 250 as respective variations of embodiments 100 and 150 of FIG. 1 and FIG. 2 while using the popular ST connectors mentioned above. Specifically, FIG. 3 shows an ST connector 204 that has two mating components 204A and 204B. Connector mating component or simply connector component 204B is rotated over connector component 204A affixed to or integrated with needle body 208 as shown. Component 204B is rotated over component 204A until the two are latched securely with the help of a locking ferrule/pin 204C. This action is reminiscent of a coaxial cable connector of cable television.

Connector 204 also has a sleeve 204D for gently guiding fiber optic cable 202 to and from the instant hypodermic needle. Fiber optic 202 is connected to a laser source 220 as shown. The rest of the elements of embodiment 200 of FIG. 3 are analogous to those of embodiment 100 of FIG. 1. More specifically, there is a needle body 208 comprising a lens housing 206 embedding a focusing lens 210 as shown. Needle body 108 also comprises a needle-hub containing members 214, 216 and any other members above shaft 218A not explicitly marked.

In a similar fashion, FIG. 4 shows embodiment 250 with the difference from FIG. 3 that needle shaft 218B has two bends as shown. Per above, shaft 218B can be of any length or diameter and may have any number of angles of bends to satisfy the needs of an application. Preferably, such bends have an angle in the range of 0 to 179 degrees. FIG. 4 also explicitly shows an imaging system 230 of the above discussion to aid/help in guiding laser-delivery needle 250 to the target site.

In related variations, instant needle body 108/208 of FIG. 1-4 consists only of a standard needle-hub. In such variations, a specialized fiber optic connector directly attaches to the hub of a standard off-the-shelf hypodermic needle via. In these embodiments, the fiber optic cable carrying laser radiation feeds into a specialized or modified connector that connects directly to a standard hypodermic needle. Various kinds of such specialized/modified connectors may be made that are designed to match the mating connections of hypodermic needles that are presently available or to be designed in the future. Such mating connections include but are not limited to a luer lock, a slip-tip connector, a catheter tip connector, a threaded connector and an eccentric tip connector.

Alternatively, these embodiments utilize an adapter of the present technology that connects to a standard optical connector carrying an optical fiber at one end, and to a standard hypodermic needle at the other end. These embodiments utilizing standard hypodermic needles may or may not utilize a focusing lens and are discussed further below.

Thus, in a highly preferred set of embodiments, the lens is embedded in an adapter that is designed specifically for a given type of optical connector. The adapter preferably contains a lens for focusing the laser radiation into a needle shaft. The adapter then attaches to a standard off-the-shelf hypodermic needle for the delivery of laser radiation to the target site. The advantage of these embodiments is that adapters for various types of optical connectors can be built within the scope of the present principles. These adapters connect to an optical fiber via an optical connector at one end and to a standard hypodermic needle at the other end.

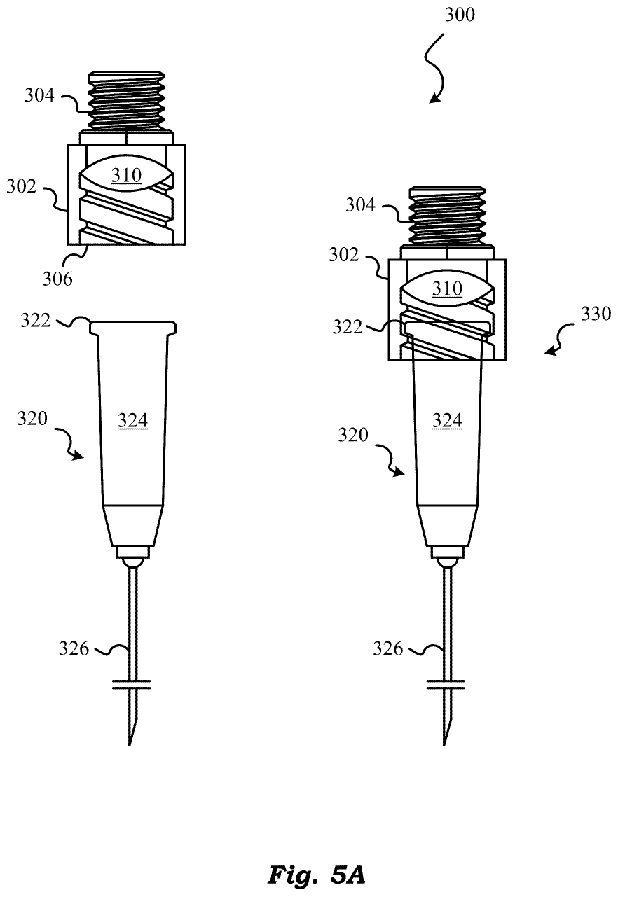

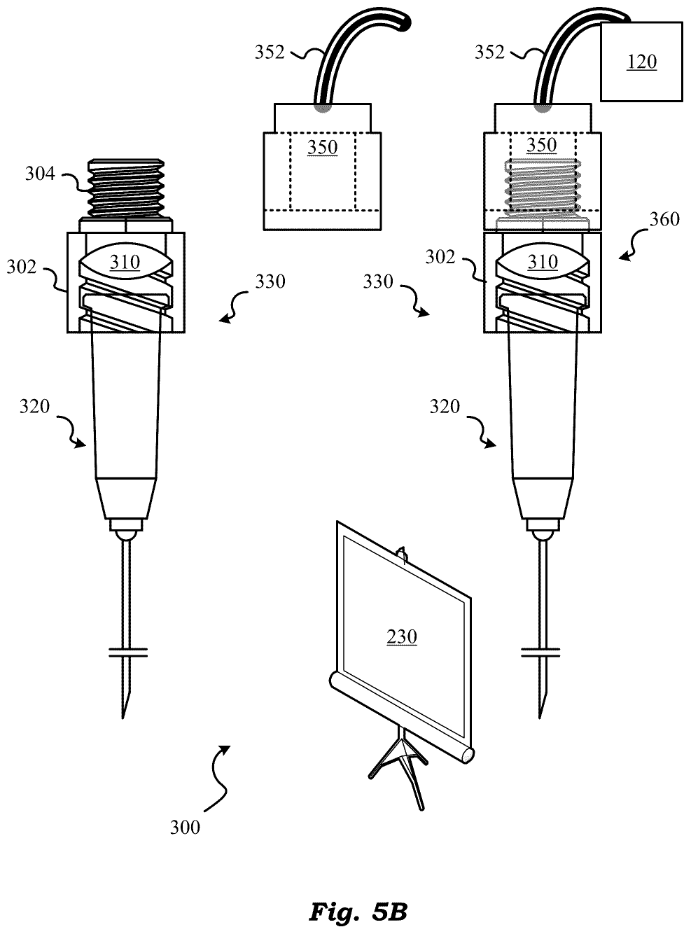

FIG. 5A and FIG. 5B show one such embodiment 300 with an adapter 302. Adapter 302 is exemplarily designed to connect to an SMA fiber optic connector introduced earlier. More specifically, instant adapter 302 contains a lens 310 embedded in and has a female SMA connector 304 at its upper end. Adapter 302 also has a luer lock connector 306 at its lower end that rotates around and fits over a luer lock 322 of a standard hypodermic needle 320 as shown. Standard hypodermic needle 320 also has a needle-hub 324 and a shaft 326 as shown.

Once adapter 304 rotates and attaches to luer lock 322 of hypodermic needle 320 on the left-hand side of FIG. 5A, we obtain configuration 330 shown on the right-hand side of FIG. 5A. Specifically, we obtain an instant adapter 302 with an SMA connector 304 attached to a standard hypodermic needle 320 via luer lock 322. Now, our female SMA connector 304 can receive a male counterpart SMA connector whose distal end carries an optical fiber 352 as shown in FIG. 5B.

More specifically, FIG. 5B shows configuration 330 of FIG. 5A and a male SMA connector 350 attached to or carrying an optical fiber or fiber optic 352. Note that not all reference numerals of configuration 330 from FIG. 5A are shown in FIG. 5B to avoid clutter. Now, male SMA connector 350 is attached to female SMA connector 304 by rotating it around the threads of female SMA connector 304 as shown. We thus finally obtain configuration 360 of our instant laser-delivery hypodermic needle that has an instant adapter 302. Adapter 302 is attached to a standard hypodermic needle 320 at one end and to an SMA optical connector 350 carrying an optical fiber 352 at the other end.

FIG. 5B also explicitly shows laser source 120 and imaging or guidance system 230 from the prior embodiments that are omitted in some drawing figures to avoid clutter. In the example shown in FIG. 5A-B, our adapter 302 has a lens 310 although this is not a requirement. In other words, a lens is preferable because it allows focusing of the laser radiation into the needle shaft so that more effective energy transfer from the laser radiation to the target site can take place. Lens 310 in combination with adapter 302 also act as a seal against contamination of the target site by preventing any bodily fluids from coming in contact with the upstream components.

In variations of the present design that do not have a lens, laser energy is still usefully transferred to the target site, but potentially less efficiently. Furthermore, in the example of FIG. 5A-5B a luer lock 322 is used to attach adapter 302 to needle 320, although that is also not a requirement. Luer lock is a popular locking mechanism for hypodermic needles, although it is one of the many locking mechanisms that the present technology can employ to attach adapter 302 to needle 320. Other exemplary locking mechanisms include but are not limited to a slip-tip connector, a catheter tip connector, a threaded connector and an eccentric tip connector.

Furthermore, the present technology does not prescribe a specific order in which various parts of final configuration 360 of laser-delivery hypodermic needle shown in FIG. 5B need to be attached. In other words, alternatively to the attachment order discussed above, it is possible that male SMA connector 350 is first attached to its female counterpart 304, before underlying instant adapter 302 is attached to needle 320. The modular design of the laser-delivery techniques taught herein allows a practitioner to quickly and securely connect and disconnect various parts of the instant hypodermic needles, including its fiber optic connections.

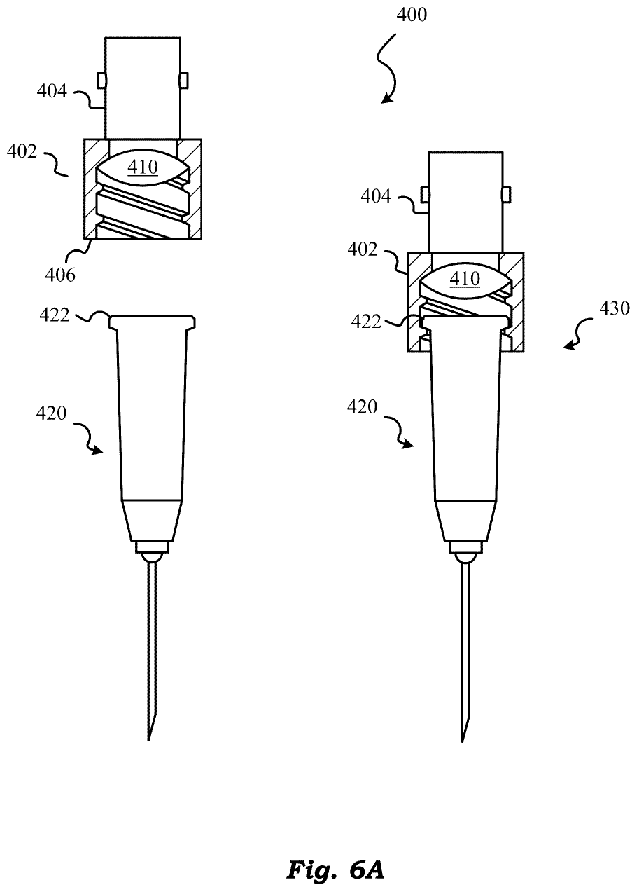

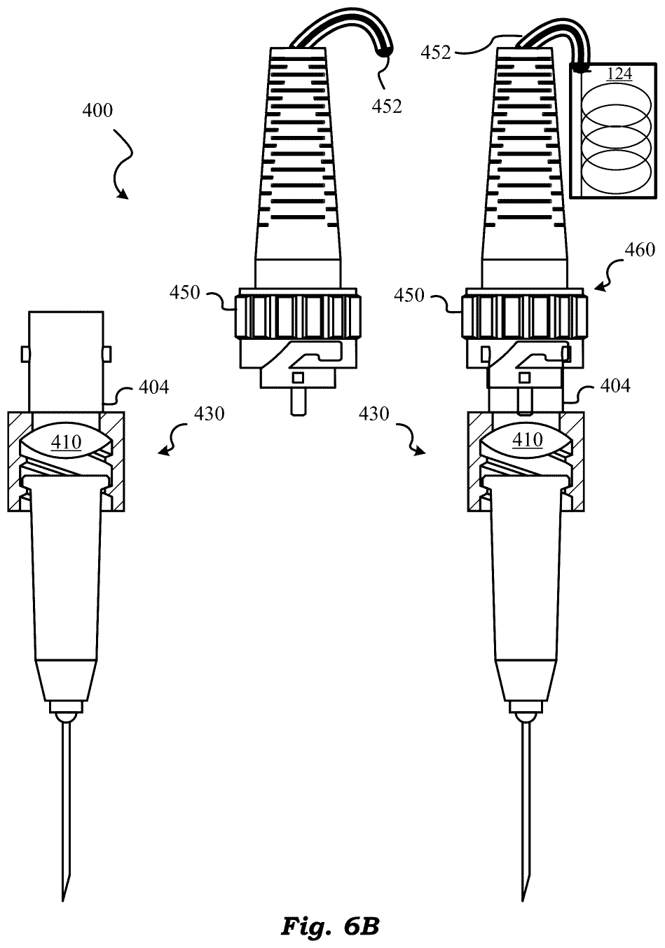

In the example of FIG. 5A-5B our adapter 302 is specifically designed to accommodate an SMA fiber optic connector. SMA connectors are just one of the many connector types that the present design can support as taught throughout this specification. Thus, a variation 400 of the present embodiments utilizes another popular fiber optic connector i.e. the ST connector introduced earlier. Such a variation is shown in FIG. 6A and FIG. 6B.

In a manner analogous to embodiment 300 of FIG. 5A, FIG. 6A shows an embodiment 400 with an adapter 402 based on the instant principles. Adapter 402 has a lens 410 for focusing laser radiation, and has a female ST connector 404 at its upper end as shown. It also has a luer lock connector 406 at its lower end as shown. FIG. 6A also shows a standard hypodermic needle 420 with not all its parts marked with reference numerals as needle 320 of FIG. 5A-B to avoid clutter and repetition. Needle 420 has a luer lock 422 as shown. As in the prior embodiments, adapter 402 attaches to luer lock 422 of needle 420 to obtain an intermediate configuration 430 as shown in FIG. 6A.

FIG. 6B shows configuration 430 of FIG. 6A and a male ST connector 450 carrying an optical fiber 452. Male ST connector 450 attaches to its female counterpart 404 as shown to obtain final configuration 460 of our laser-delivery hypodermic needle of the present principles. FIG. 6B also shows a laser source 124 which is a fiber laser providing laser radiation to laser-delivery hypodermic needle 460 via fiber optic 452.

As in other embodiments, an imaging or guidance system is also preferably utilized to assist an operator in guiding the instant laser-delivery hypodermic needle(s) to the target site(s) per above discussion. The imaging/guidance system is not explicitly shown in FIG. 6, and as before with the drawing figures of FIG. 5A-B, not all reference numerals for all configurations of FIG. 6A-B are repeated to avoid clutter and for clarity of explanation.

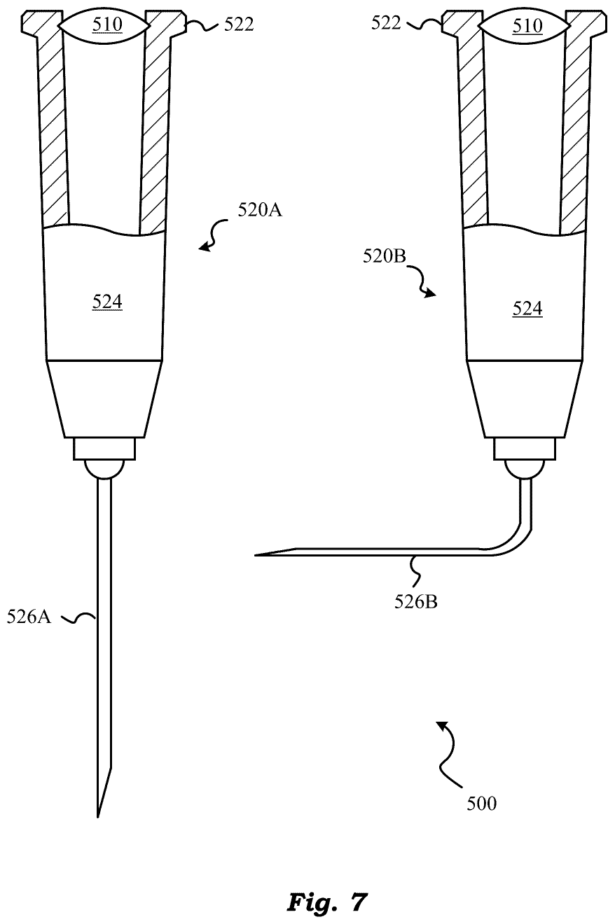

FIG. 7 shows yet another set of embodiments 500 where a standard hypodermic needle with a luer lock has a lens embedded in it or integrated with it. More specifically, FIG. 7 shows a modified hypodermic needle 520A that has a lens 510 embedded/integrated with/in it. Modified needle 520A of the instant design has a straight shaft 526A, a hub 524 and a luer lock 522. A variation 520B has an angled shaft 526B as shown. In the present embodiments, any off-the-shelf or standard fiber optic connector that has or attached to a luer lock adapter can now be attached to our modified needles 520A-B.

Thus, the present variation allows for minimal modifications of standard hypodermic needles to accrue the benefits of the delivery of pulsed laser radiation to the target site(s). Modified needles 520A-B may be single-use and disposable or sterilized for a given number of uses. These and other embodiments may also benefit from single-mold manufacturing techniques to reduce cost of production. Using single-mold manufacturing, instant laser-delivery hypodermic needles may be manufactured as a single or one-piece item, and may thus be more amenable for being disposable after each use—just like standard hypodermic needles.

In related variations, lens 510 may not exist at all. In still other variations, instead of lens 510 there may be a flat glass window or sheet covering the entrance to the needle-hub. Such variations may not benefit from the focusing ability of the lens and consequently may have comparatively less efficient transfer/delivery of laser energy to the target site. However, they are still operational/functional and suitable for certain applications.

Aside from focusing laser radiation into shaft 526, lens 510 also acts a seal and prevents contamination of the apparatus and/or the target site by stopping any bodily fluid from the target site(s) of the organism from coming in contact with the fiber optic connector or other upstream components. In variations that utilize a glass window instead of a lens as discussed above, the contamination is prevented by the glass window. In fact, in order to serve the needs of a given application, a glass window may be used instead of a focusing lens in any of the embodiments taught herein. As further stated, depending on the variation, there may not be either a lens or a glass window in any of the embodiments taught herein.

However, in still other variations, needles 520A-B may not have any modification done to them whatsoever. Such variations can thus utilize fully off-the-shelf components including a fiber optic connector that can fit a standard hypodermic needle for delivering laser radiation to a target site based on the instant principles. Of course, such variations will not have the advantage of laser beam focusing afforded by a lens or lenses as well as the contamination prevention abilities of a lens or a glass window or an instant adapter. However, they may still serve practical use-cases where standard optical and medical parts/components are the only option.

In another highly interesting set of embodiments of the present technology, the source of laser radiation, which may not be explicitly shown in all drawing figures but is presumed to exist, is integrated with the instant laser-delivery hypodermic needle.

In such “self-contained” variations, the laser source is powered by a battery that is also integrated with or embedded into the instant self-contained or single piece/unit laser-delivery hypodermic needle.

Exemplary laser sources that can be used for these embodiments include laser diodes because they are solid-state and have a small enough form factor. Moreover, the laser diode is connected to and controlled by a wireless adapter controller that is also embedded in or integrated with our self-contained wireless laser-delivery hypodermic needle. The wireless adapter/controller may utilize one or more of the many available wireless technologies available in the industry, including but not limited to, Wifi, Bluetooth, Near Field Communication (NFC), ZigBee, etc. The wireless adapter/controller onboard the laser-delivery device thus communicates with its remote counterpart to receive instructions on how to deliver laser radiation to the target site.

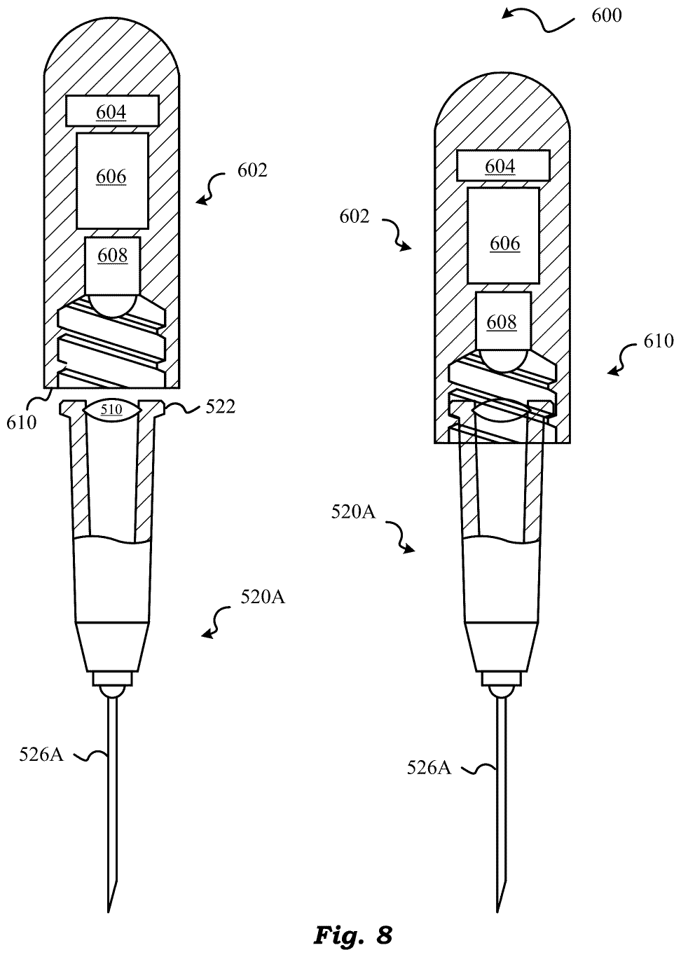

FIG. 8 shows an example of the present embodiments. More specifically, in exemplary embodiment 600 shown in FIG. 8, there is a hypodermic needle 520A with a lens 510 and luer lock 522 from the embodiments of FIG. 7A-B. There is also a self-powered or self-contained wireless laser cap or housing 602 as shown. Wireless laser housing 602 has a battery 604 that is used to power a wireless controller 606 and a laser diode 608 onboard as shown. Housing 602 has a luer lock connector 610 that attaches to luer lock 522 of modified needle 520A to obtain configuration or unit 610 of our self-contained wireless laser-delivery hypodermic needle. Again, not all parts of configuration/unit 610 may be marked explicitly to avoid clutter and repetition.

Analogously to prior embodiments, in related variations of the present embodiments, instead of (or in addition to) lens 510 there may be a just a glass window to prevent contamination. Still alternatively, there may not be a lens or window at all and needle 520A may be a standard off-the-shelf hypodermic needle with no modifications whatsoever. In still other variations, lens 510 may be embedded in housing 602 itself rather than needle 520A which may thus be a standard unmodified hypodermic needle.

Of course, luer lock 522 and luer lock connector 610 are just one of the available locking mechanisms for hypodermic needles and housing 602 may be adapted to lock onto any locking mechanism for hypodermic needle 520A within the scope of the present principles.

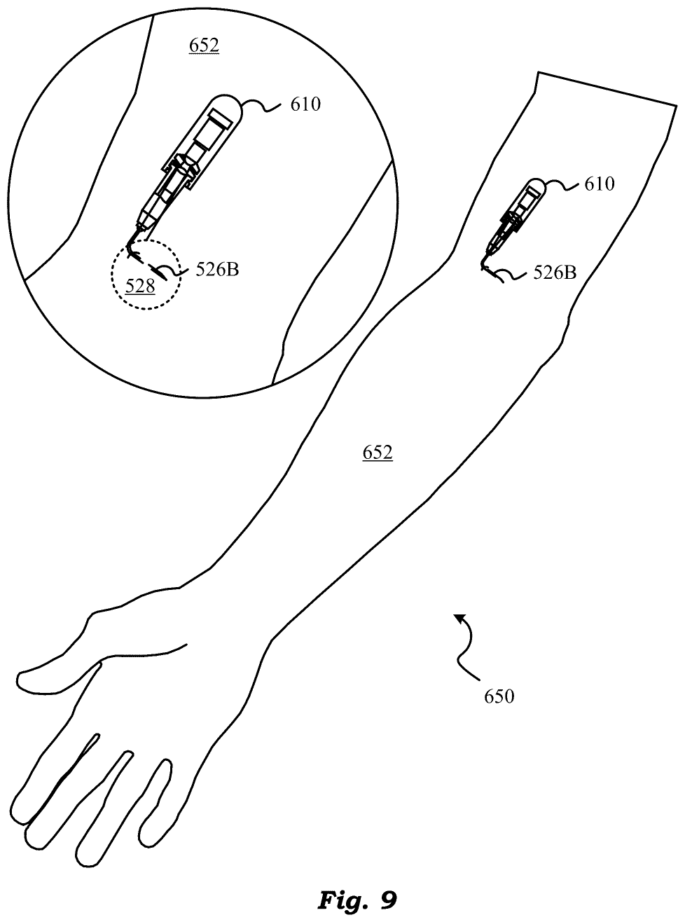

There are many practical advantages obtained from the flexibility of design of the present embodiments. In one such exemplary use-case, our self-contained laser-delivery hypodermic needle with its form/configuration 610 as shown in FIG. 8, is used as an indwelling or wearable device. Such an indwelling/wearable device or unit is applied to an organism, who may exemplarily be a patient. An indwelling or wearable application 650 of the present variations is shown in FIG. 9.

FIG. 9 shows an arm 652 of a human, along with its exploded view. The arm has an attached self-contained instant laser hypodermic needle in its configuration 610 of FIG. 9, or simply put our instant laser-delivery hypodermic needle 610. Instant hypodermic needle 610 is used as a wearable device by the organism that may remain attached to the organism/patient for an extended period of time. Per above, indwelling/wearable device 610 of the instant design is battery-powered and contains a wirelessly controlled laser diode integrated with a hypodermic injection/needle. The needle shown in FIG. 9 on arm 652 has a bent/angled shaft 526B and is used to deliver pulsed laser radiation to a target site 528 in arm 652 as shown.

In the present embodiments, the amount of radiation and length of dosage can be varied and controlled wirelessly. If the laser radiation is pulsed, then the period of the pulsed laser radiation i.e. the time period between the pulses, as well as the length of the pulses can also be wirelessly controlled. Unit 610 can shut off or be changed to a different setting at the programmed time intervals. The patient can return to the treatment center for additional treatment or removal of unit 610. This use-case may be suitable for a variety of purposes including optogenetics treatment, laser therapy, etc.

In another exemplary use-case, the self-contained battery powered unit 610 of the embodiments of FIG. 8-9 is used as an acupuncture needle for acupuncture treatment. For acupuncture treatment, the needle shaft has dimensions suitable for acupuncture. That means, that shaft diameter is preferably of a micro-gauge i.e. preferably in the range of 0.12-0.35 millimeters. That also means that the length of the shaft is in accordance with the diameter and as dictated by the type of acupuncture treatment and target site(s).

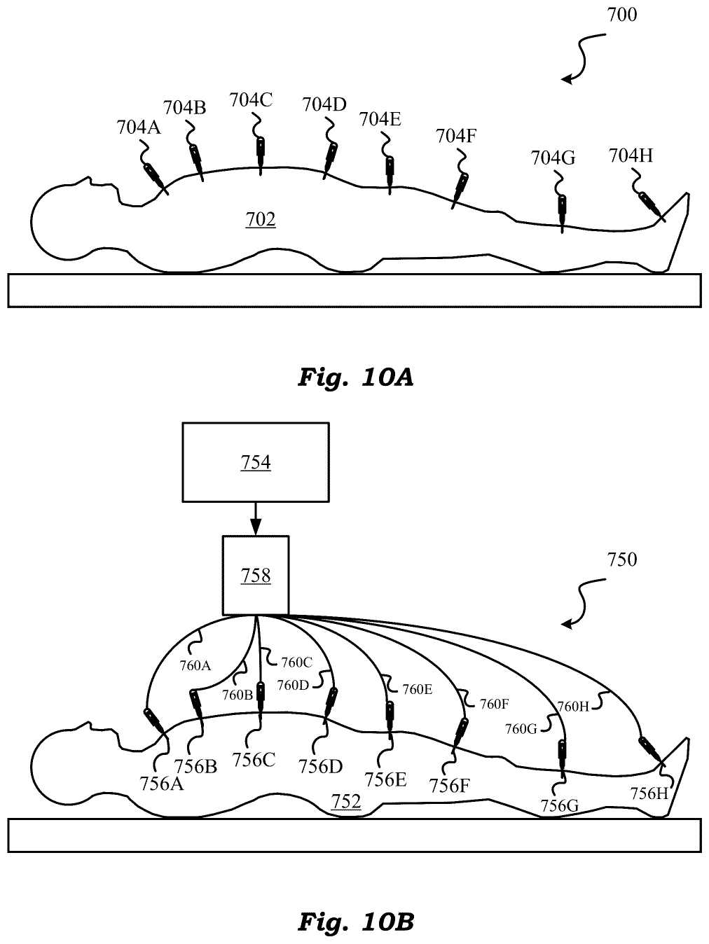

One such exemplary acupuncture application 700 is shown in FIG. 10A. FIG. 10A shows an organism who is a human patient 702. There any desired number of instant self-contained battery powered units 704 of the above teachings inserted into patient 702 at the desired target sites in/on the patient's body as shown. FIG. 10A shows 8 such instant laser-delivery acupuncture needles/devices 704A, 704B, 704C, 704D, 704E, 704F, 704G and 704H inserted at corresponding target sites in the body of patient 702.

In related variations, it is also possible that the acupuncture treatment based on the present technology utilizes wired or cabled laser-delivery needles of the embodiments of FIG. 1-7 rather than the wireless embodiments of FIG. 8-9. Such a wired/cabled acupuncture embodiment 750 is also shown in FIG. 10B for comparative purposes. More specifically, FIG. 10B shows a patient 752 with 8 instant laser-delivery acupuncture needles 756A-H from the embodiments of FIG. 1-7 inserted/attached. These needles are being provided with laser radiation from a suitable laser source 754.

In a preferred variation of the present embodiments, the laser radiation produced by laser source 754 is first sent to a beam-splitter 758 as shown. A beam-splitter as known to skilled artisans splits the incident beam into a plurality of resultant beams, each with lower power than the incident beam. In the example shown in FIG. 10B, beam-splitter 758 splits the laser beam generated by laser source 754. Each of the resulting beam from beam-splitter 758 is then carried by corresponding fiber optic cables 760A-H to respective instant acupuncture needles 756A-H for laser-delivery to respective target sites in patient 752.

It is quite possible that the amount and duration of dosage of laser radiation, as well as the length and period of any laser pulses, for the acupuncture treatment are different from other treatments. Hence, through wireless or wired means, the programming in units 704 of FIG. 10A and 756 of FIG. 10B can thus be adjusted accordingly. Of course, it is also possible that individual units 704G-H and 756G-H have different programming depending on the target site that each is attached to and depending on the application. It is also possible, that instant needles 704A-H and 756A-H are simultaneously performing different treatments including acupuncture, therapy, rejuvenation, optogenetics, etc.

Of course, it is also possible to incorporate wireless technology for controlling the delivered laser radiation even in the cabled laser-delivery embodiments. What this means in such variations is that while the laser energy is transmitted from a separate laser source via fiber optic cables, it is controlled by a wireless controller that is onboard the instant needle. As in any other embodiments, any loss of power through the needle can be calibrated and compensated for to deliver the proper dosage at the output.

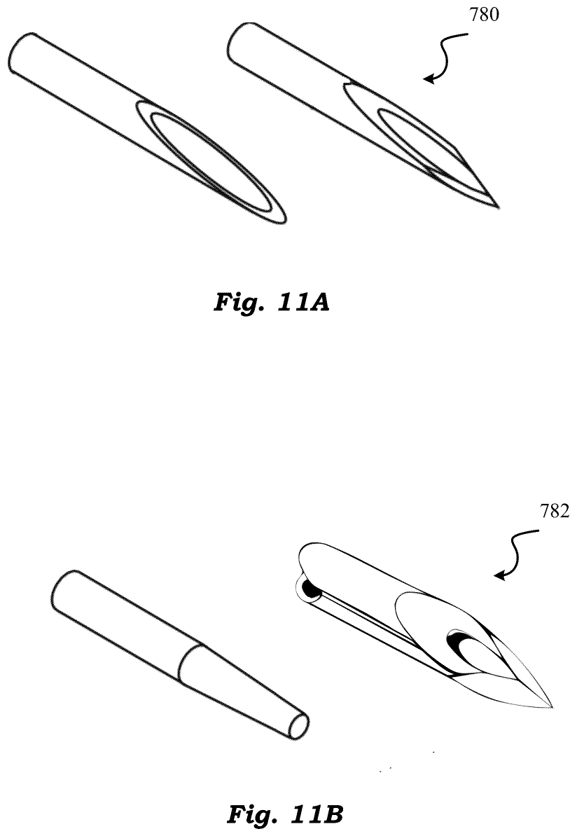

In any of the above taught embodiments, the bevel of the hypodermic needle may be adapted to suit the needs of a given application. For example, the bevel end of the needle shaft may be axisymmetric, in which case the laser radiation emanating from the bevel has an omnidirectional radiation pattern. Alternatively, the bevel is asymmetric, in which case the laser radiation emanating from the bevel has a unidirectional pattern. FIG. 11A explicitly shows two exemplary asymmetric bevels 780 and FIG. 11B shows two exemplary axisymmetric bevels 782 that can be used to provide unidirectional and omnidirectional laser delivery to target site(s) respectively.

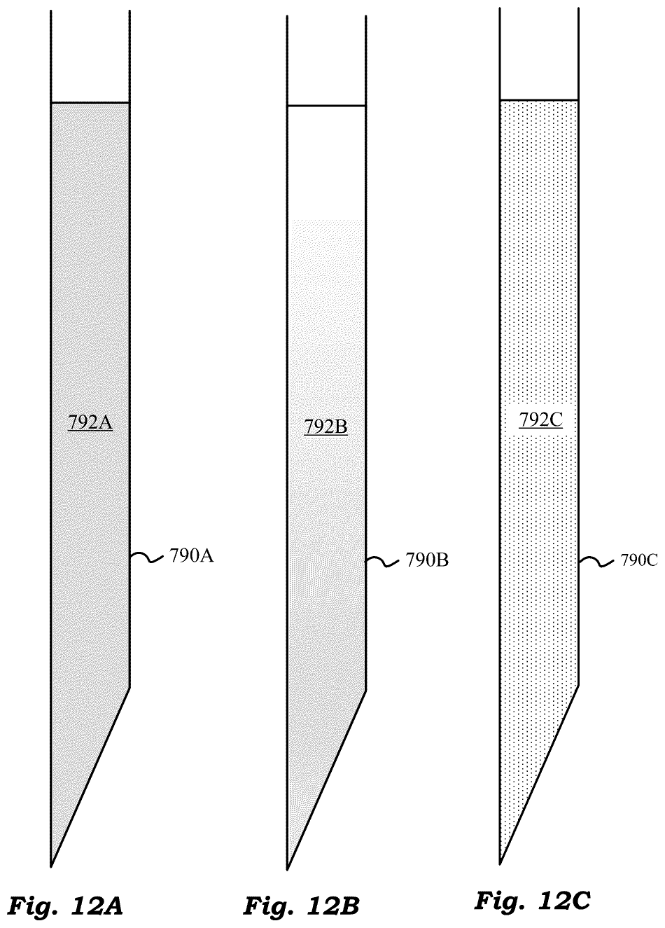

As noted above, the laser shaft may have a filling of a suitable material, including a glass filling, a polymer filling or the filling may just be that of bodily fluid that backfills or travels up the hollow needle shaft of an instant laser-delivery hypodermic needle during treatment. In any case, the filling needs to be transmissive enough for the wavelength of laser to propagate through it and deliver sufficient laser energy to the target site. FIG. 12A shows an instant needle shaft 790A with a polymer filling 792A. FIG. 12B shows an instant needle shaft 790B with a glass filling 792B, and FIG. 12C shows an instant needle shaft 790C with a bodily fluid filling 792C.

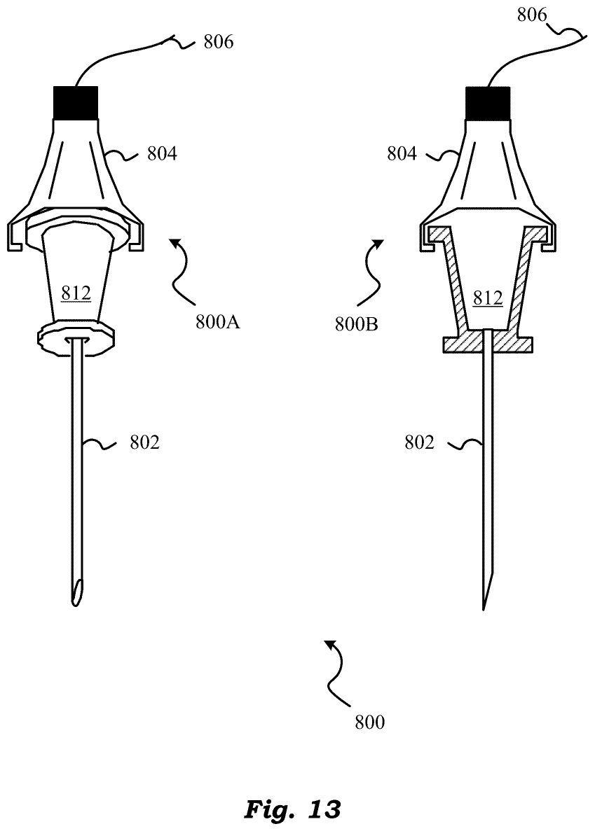

For completeness, FIG. 13 shows two views of an adapter-less variation of the embodiments discussed above. More specifically, FIG. 13 shows a normal view 800A and a cross-sectional view 800B of an embodiment 800 that uses a standard hypodermic needle. The standard hypodermic needle has a hub 812 and a shaft 802. Hub 812 attaches to a specialized or modified optical connector 804 of the instant design that is carrying a fiber optic cable 806. The connection between instant modified/specialized connector 804 and the standard hypodermic needle is preferably a luer lock connection as shown, although it may be any type of suitable mating connection. The variation shown in FIG. 13 thus allows for off-the-shelf components to accrue the benefits of the present design with minimal changes.

The present techniques further allow for instant laser-delivery hypodermic needles to be shaped in forms of a variety of medical instruments and devices, including but not limited to hypodermic needles as taught above, blood lancets, cannulas, catheters, etc.

Aside from the various applications mentioned above, there are a number of treatments possible by the instant technology that were not available in the prior art. These include but are not limited to:
Tumors, cysts, and polyps, etc. anywhere in the body can now be treated by inserting/injecting an instant laser-delivery hypodermic needle directly into it. The laser energy is then delivered directly into the tumor, cysts, polyps, etc. destroying it without damaging any surrounding tissue. Blood vessels are also cauterized by the laser energy which blocks any metastasizing through blood flow. Inoperable tumors can now be treated with the present techniques.
Using the present laser-delivery devices/needles, laser energy can be delivered directly to any part of a joint or cartilage, in order to stimulate healing and to reduce inflammation. This is a vast improvement over surgery, or indirect methods of applying laser energy externally to the body and relying on deep tissue penetration to reach the target site.
Using the present techniques, laser energy can now be delivered/applied directly to herniated or bulging discs to stimulate healing and to reduce inflammation. This is a vast improvement over surgery, or indirect methods of applying laser energy externally to the body and relying on deep tissue penetration to reach the target site.
Using the present techniques, laser energy can now be delivered exactly where needed for the rapidly growing field of optogenetics, without need for surgery or optical implants.
Based on the present design, laser energy can now be delivered to acupuncture points throughout the body to enhance and amplify the effects of acupuncture and to distribute the rejuvenating effects of the laser throughout the body. This can have the effect of reversing biological aging.
Using the present techniques, all cells, tissues, and organs can be rejuvenated by direct laser stimulation without need for surgery or other invasive methods.
Many interesting and useful ways of delivering the laser energy can be conceived based on the present techniques. These include delivering the laser energy while the needle is rotated in place, drawn through the body, and rotated while drawn through the body.
The present techniques can be applied with laser sources that generate laser radiation across a wide spectrum of electromagnetic wavelengths. Furthermore, while special emphasis is paid to laser radiation in the above-taught embodiments, as noted above, these techniques can also be applied to non-laser or non-coherent electromagnetic radiation.
The present techniques can also supplement other existing techniques such as stem cell therapy to enhance and augment the outcome.

In view of the above teaching, a person skilled in the art will recognize that the methods of present invention can be embodied in many different ways in addition to those described without departing from the principles of the invention. Therefore, the scope of the invention should be judged in view of the appended claims and their legal equivalents.

What is claimed is:

1. An apparatus comprising:
(a) a pulsed laser source for generating pulsed laser radiation;
(b) an optical fiber for transmitting said pulsed laser radiation from said pulsed laser source to a body of a hypodermic needle;
(c) a lens embedded in said body for focusing said pulsed laser radiation into a shaft of said hypodermic needle; and
(d) an imaging system for guiding said hypodermic needle towards a target site in an organism;
wherein said pulsed laser radiation light-pipes through said shaft and is incident on said target site.

2. The apparatus of claim 1 wherein said optical fiber is connected to said body via an adapter.

3. The apparatus of claim 1 wherein said pulsed laser radiation light-pipes through a filling inside said shaft of said hypodermic needle, said filling comprising a material including a bodily fluid of said organism, a polymer and glass.

4. The apparatus of claim 1 wherein said shaft is internally polished to facilitate total internal reflection of said pulsed laser radiation that light-pipes through it.

5. The apparatus of claim 1 wherein said optical fiber connects to said body via one of a bayonet connector, a friction lock, a magnetic connector, a SubMiniature version A (SMA) Connector, a Straight Tip (ST) connector, a Subscriber Connector (SC), a Miniature Unit (MU) connector, a Fiber Channel (FC) connector, a Mechanical Transfer Registered Jack (MTRJ) connector, a Lucent Connector (LC) and an E2000 connector.

6. The apparatus of claim 1, wherein said shaft is bent at an angle in the range of 0 to 179 degrees.

7. The apparatus of claim 1 having one of an asymmetric bevel and an axisymmetric bevel for producing a unidirectional pattern and an omnidirectional pattern respectively of said pulsed laser radiation incident on said target site.

8. The apparatus of claim 1, wherein said shaft has a micro-gauge diameter suitable for an acupuncture treatment.

9. The apparatus of claim 1 wherein said target site comprises an internal organ, a tumor, a cyst, a polyps, a joint, a cartilage, a spinal cord disc, one or more stem cells, an acupuncture site and an optogenetic treatment site.

10. A wearable laser-delivery hypodermic needle integrated with a solid-state pulsed laser source which generates pulsed laser radiation that light-pipes through a filling inside a shaft of said laser-delivery wearable hypodermic needle and is delivered to a target site in an organism.

11. The wearable laser-delivery hypodermic needle of claim 10 manufactured as a disposable device.

12. The wearable laser-delivery hypodermic needle of claim 10, wherein said pulsed laser radiation is controlled wirelessly.

13. The wearable laser-delivery hypodermic needle of claim 10 used for acupuncture treatment.

14. A method of delivering pulsed laser radiation to a plurality of target sites in an organism, said method comprising the steps of:
(a) operably connecting each of a fiber optic cable from amongst a respective plurality of fiber optic cables, to a corresponding hypodermic needle from amongst a respective plurality of hypodermic needles, for carrying said pulsed laser radiation;
(b) directing said corresponding hypodermic needle towards a corresponding target site from amongst said plurality of target sites, by inserting it into said organism;
(c) focusing said pulsed radiation into a shaft of said corresponding hypodermic needle by a lens embedded in said corresponding hypodermic needle;
(d) light-piping said pulsed radiation through said shaft for said delivering; and
(e) preventing a contamination of said corresponding target site from amongst said plurality of target sites by a filling inside said shaft.

15. The method of claim 14 first passing said pulsed laser radiation through a beam splitter where said beam splitter produces a plurality of laser beams of said pulsed laser radiation, each of said plurality of laser beams carried by one of said respective plurality of fiber optic cables in said step (a).

16. The method of claim 15 providing one or more of said plurality of target sites to be an acupuncture site.

17. The method of claim 14 providing said laser source to be a fiber laser.

18. A method of delivering pulsed laser radiation to a target site in an interior of an organism, said method comprising the steps of:
(a) generating said pulsed laser radiation by a solid-state laser source integrated with a hypodermic needle;
(b) directing said hypodermic needle towards said target site by inserting it into said organism;
(c) focusing said pulsed laser radiation into a shaft of said hypodermic needle by one or more lenses embedded in said hypodermic needle;
(d) light-piping said pulsed laser radiation through said shaft for said delivering;
(e) preventing a contamination of said target site by said one or more lenses; and
(f) wirelessly controlling said delivering of said pulsed laser radiation to said target site.

19. The method of claim 18 performing said directing in said step (b) with the aid of one or both an online imaging system and an offline imaging system.

20. The method of claim 18 utilizing said hypodermic needle integrated with said solid-state pulsed laser source, as a disposable device.

21. The method of claim 18 providing said solid-state laser source to be a laser diode.

22. The method of claim 21 utilizing said solid-state laser source integrated with said hypodermic needle, as an indwelling device.
